(19) United States
(12) Patent Application Publication
(10) Pub. No.: US 2021/0059244 A1
(43) Pub. Date: Mar. 4, 2021

(54) CLAY-BASED CARRIER PLATFORM FOR BIOLOGICALS IN AGRICULTURE

(71) Applicant: OIL-DRI CORPORATION OF AMERICA, Chicago, IL (US)

(72) Inventors: Sajo P. Naik, Naperville, IL (US); Shalaka Shinde, Arlington Heights, IL (US)

(21) Appl. No.: 17/003,409

(22) Filed: Aug. 26, 2020

Related U.S. Application Data
(60) Provisional application No. 62/891,776, filed on Aug. 26, 2019.

(51) Int. Cl. A01N 25/08; A01N 63/38; A01N 63/30

(52) U.S. Cl. CPC A01N 25/08; A01N 63/30; A01N 63/38

(57) ABSTRACT

The present invention relates to an improved carrier for sustaining microbes, comprising of a composition having a base material with one or more of a clay or mineral, the composition further including a carbon source and a pH modifier. More specifically, an improved biological carrier platform is provided with scientifically customized formulations that provide hospitable environments for a variety of biologicals, including live microbes and bioactive essential oils. The platform offers an inert granular, mineral base in various grind sizes and forms having specific beneficial effects. In addition, present invention also provides the various processes and methods such as providing an improved carrier comprising a base material having one or more of a clay or mineral, the composition further comprising a carbon source and a pH modifier; and loading a microbial inoculant onto the carrier, wherein the microbial inoculant comprises a plurality of microbes.

### RELATED APPLICATIONS AND INCORPORATION BY REFERENCE

This application claims benefit of and priority to U.S. provisional patent application Ser. No. 62/891,776 filed Aug. 26, 2019. The foregoing application, and all documents cited therein or during its prosecution (“application cited documents”) and all documents cited or referenced in the application cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The present invention generally relates to biostimulants for promoting plant and root growth. More specifically, the present invention relates to an improved biological carrier platform scientifically customized and formulated to meet the specific needs of various active ingredients and related methods to successfully load biological active ingredients onto this improved carrier platform for a biostimulant and biocontrol agricultural application.

### BACKGROUND OF THE INVENTION

The agricultural industry has always recognized the importance of fertilizers and natural biostimulants in promoting plant and root growth. Biostimulants can include humic and fulvic acids, minerals, protein hydrolysates, various plant extracts, chitosan and biopolymers, and beneficial bacteria and fungi.

Soil microbes such as bacteria and fungi play a significant role in a terrestrial ecosystem. They regulate plant productivity by colonizing plants, digesting essential minerals and decomposing organic matter. The symbiotic association of these soil microbes with plant roots creates an important network of nutrient-exchange between plant and microbes, and in turn provides resistance to biotic and abiotic stresses. Consequently, these microbes (such as nitrogen fixing bacteria, rhizobium, mycorrhizal fungi, and other bacteria) have become an important part of agriculture.

To maintain crop productivity, the agriculture industry also needs to manage different insects, nematodes, and other pathogenic microbes. Over the past several years, the industry has begun to use organic materials such as leaves, bark, essential oils, or microbes to control some of the harmful insects, nematodes and pathogens. The application of these materials is categorized as biocontrol.

Current commercial biological carrier products are sold in liquid form. The limitations of these liquid formulations include stability of active ingredients, speed at which the active ingredient is released, uneconomical shipping costs due to the weight of water and application challenge such as environmental drift. The agriculture industry produces liquid, solid/granular or wet-able powder formulations containing a wide range of biostimulant and biocontrol microbes. The industry also uses granules as carriers to efficiently deliver the beneficial microbes or active ingredients to the target plant root, leaves or fruits. Several materials such as bagass, sugar cane filter mud, charcoal, straws, various compost mixtures, peat, clay, and minerals may be suitable for this purpose. Importantly, the chemical and physical properties of the material used as a carrier determines the compatibility of each carrier with a specific microbial inoculant. The use of solid, granular biological carriers can help overcome these limitations.

Commercially, peat is one of most commonly used solid carriers for Rhizobium. sp used worldwide. Other beneficial biologicals, including Pseudomonas. sp, Bacillus. sp, Glomus. sp, Pochonia. sp or Trichoderma. sp, etc. are either commercially available in liquid or powder form. However, the main obstacle for the continuous use of peat as a carrier for soil microbes is the natural variability in its composition, resulting in inconsistent performance. In addition, there are limited sources for consistent peat, leading to availability issues. Thus, there currently exists an unmet need for reliable and economical substitutes to peat as a solid carrier for agriculturally important microbes.

### SUMMARY OF THE INVENTION

This summary is provided to introduce a selection of concepts in a simplified form that are further described in the detailed description of the disclosure. This summary is not intended to identify key or essential inventive concepts of the claimed subject matter, nor is it intended for determining the scope of the claimed subject matter.

In one aspect of the present disclosure, a carrier is provided comprising a base material comprising one or more of a clay or mineral, the composition further comprising a carbon source and a pH modifier. The biological carriers can host compounds and microorganisms and various experiments show that plant extracts, essential oils and fatty acids remain functionally active and stable on the biological carriers over time. Research has also demonstrated that certain essential oils impregnated on the disclosed biological carrier maintains antimicrobial activity against pathogenic fungi and bacteria in vitro. Further, such biological carrier platform offers scientifically customized formulations that provide hospitable environments for a variety of biologicals, including live microbes and bioactive essential oils.

In another aspect of the present disclosure, the biological carrier platform offers an inert granular, mineral base in various grind sizes and forms. The products are nontoxic and eco-friendly and one of the several beneficial effects of these products is the biodiversity they add to the rhizosphere. Key characteristics of disclosed mineral base include ion-exchange properties, well-defined phyllosilicate crystal structure, physicochemical qualities and a large surface area.

In another aspect, the base material may comprise one or more of montmorillonites, calcium montmorillonites, attapulgites, palygorskites, sepiolites, kaolins, micas, zeolites, and diatomaceous earth. In another aspect, the carbon source may comprise one or more of humic acid, leonardite, biochar, seaweed, corn meal, millet, oats, grain starches, carboxymethyl celluloses, polymers, or other organic cellulose carbohydrates. In yet another aspect, the pH modifier may comprise one or more of salts of calcium, potassium, and sodium.

In still another aspect, the base material may comprise about 85-99% by weight of the total composition, the carbon source may comprise about 0%-10% by weight of the total composition, and the pH modifier may comprise about 0%-5% by weight of the total composition. In a further aspect, the composition may further comprise water, the water comprising about 0%-15% by weight of the total composition. Additionally, the base material may comprise a plurality of particles, wherein each of the plurality of particles has a particle size ranging between 30 and 500 microns. Also, the composition may comprise a powder or a plurality of pellets.

In yet another aspect of the present disclosure, a method of sustaining microbes is provided, comprising the steps of providing a carrier comprising a base material comprising one or more of a clay or mineral, the composition further comprising a carbon source and a pH modifier and loading a microbial inoculant onto the carrier, wherein the microbial inoculant comprises a plurality of microbes.

In another aspect of the method, the microbial inoculant may comprise a fully grown broth. In another aspect, the microbial inoculant may comprise microbial propagules.

Accordingly, it is an object of the invention to not encompass within the invention any previously known product, process of making the product, method of using the product, or method of treatment such that Applicants reserve the right and hereby disclaim any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclaim any previously described product, process of making the product, or method of using the product.

It is noted that in this disclosure and particularly in the claims and/or paragraphs the terms such as “comprises,” “comprised,” “comprising,” and the like which is synonymous with “including,” “containing,” or “characterized by,” is inclusive or open-ended and does not exclude additional, unrecited elements or method steps; and the terms such as “consisting essentially of” and “consists essentially of” limits the scope of a claim to the specified materials or steps and those that do not materially affect the basic and novel characteristic(s) of the claimed invention.

Further areas of applicability will become apparent from the description provided herein. The description and specific examples in this summary are intended for purposes of illustration only and are not intended to limit the scope of the present disclosure. These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 is an image summarizing the results of an experiment demonstrating the efficacy of a clay-based hybrid carrier in accordance with the present disclosure.

FIG. 2 is an image summarizing the results of an experiment demonstrating the efficacy of a clay-based hybrid carrier in accordance with the present disclosure.

FIG. 3 is an image summarizing the results of an experiment demonstrating the efficacy of a clay-based hybrid carrier in accordance with the present disclosure.

FIG. 4 is an image summarizing the results of a field experiment using a clay-based hybrid carrier while growing pea/lentil in accordance with the present disclosure.

FIG. 5 is an image summarizing the results of a field experiment using a clay-based hybrid carrier while growing soybean in accordance with the present disclosure.

FIG. 6 is an image summarizing the results of a field experiment using a clay-based hybrid carrier while growing pea/lentil in accordance with the present disclosure.

FIG. 7 is an image summarizing the results of a field experiment using a clay-based hybrid carrier while growing soybean in accordance with the present disclosure.

FIG. 8 is an image summarizing the results of an experiment demonstrating the stability of a fungi in a clay-based hybrid carrier in accordance with the present disclosure.

FIG. 9A illustrates an exemplary manufacturing process and method used to make a clay-based hybrid carrier in accordance with the present disclosure.

FIG. 9B illustrates an exemplary manufacturing process and method used to make a clay-based hybrid carrier in accordance with the present disclosure.

FIG. 9C illustrates an exemplary manufacturing process and method used to make a clay-based hybrid carrier in accordance with the present disclosure.

### DETAILED DESCRIPTION OF THE INVENTION

In an embodiment, a functionalized clay-based hybrid carrier has been developed as a substitute for peat as a carrier for sustaining microbes and extending shelf lives. Generally, the clay-based hybrid carrier involves the use of a clay or mineral as a base material along with a carbon source and a pH modifier (limestone, potassium carbonate, soda ash, etc.), although additional or different ingredients may be used such as polymers or a stabilizer etc. In this embodiment, the carbon source may include one or more the following: humic acid, leonardite, biochar, seaweed, corn meal, millet, oats, grain starches, carboxymethyl celluloses, polymers, or other organic cellulose carbohydrates. However, other carbon sources are contemplated.

In addition, in this embodiment, the pH modifier may include one or more of the following: salts of calcium, potassium, and sodium, etc., although other pH modifiers may be used. Further in this embodiment, the clay may include one or more of the following: montmorillonites, calcium montmorillonites, attapulgites, palygorskites, sepiolites, and kaolins. Alternatively or in addition to the clay, other minerals may be used, including but not limited to micas, zeolites, and diatomaceous earth. This embodiment provides a consistent and stable platform as a carrier for beneficial microbes. In this embodiment, a beneficial microbial inoculant in the form of fully grown broth or microbial propagules may be loaded onto the functionalized clay-based carrier. The clay-based carrier provides a suitable environment for the inoculated beneficial microbes for an extended period of 2-3 years.

The embodiment described above may have a variety of composition while still being effective for its intended purpose. However, the inventors have determined the following ranges by weight of composition for each of the three main ingredients is as follows: the clay or mineral may be about 85%-99% by weight of the total composition, the carbon source may be about 0.5%-10% by weight of the total composition, water may be about 0-15% by weight of the total composition, and the pH modifier may be about 0-5% by weight of the total composition.

Further, the embodiment described above may have a variety of composition while still being effective for its intended purpose. However, the inventors have determined that the following characteristics of the clay/mineral sources may be preferable. The particle size of the clay/mineral sources may range between 30 microns (400 Mesh) to 5000 microns (4 Mesh). The processing of the clay varies depending on the level of moisture desired in the clay after the drying process. The moisture level is referred to as regular volatile moisture (RVM) or low volatile moisture (LVM). For example, dry RVM clay granules can be superheated to make LVM products at temperatures less than 400° C. (˜750° F.). In addition, the clay/mineral sources may have a free moisture ranging from 4%-20% (RVM or Regular Volatile Material) or a range from 0%-4% (LVM or Low Volatile Material). Further, while the clay/mineral sources of this embodiment may be in a variety of shapes and sizes, agglomerated granules and extruded pellets of the clay/mineral sources may be used.

In a preferred embodiment, the clay source may be a 12/40 mesh LVM-MS Calcium Montmorillonite, the carbon source may be humic acid, and the pH modifier may be CaCO₃ powder. Also in a preferred embodiment, the clay-based hybrid carrier may include about 88.5%-92% by weight of the clay or mineral, about 2.5%-5% by weight of the carbon source, about 0.5%-1.5% by weight of a pH modifier, and about 2.5-5.5% by weight of water.

In another preferred embodiment, the composition may include a combination of clay and millet flour. The composition may include about 80-90% by weight of the clay, 0.5-4% by weight of millet flour, and about 8-13% by weight of water. The clay may be any number of clays, but in this embodiment is 24/48 Taft clay.

In order to determine the efficacy of the clay-based hybrid carrier, the inventors conducted several experiments. Clay-based hybrid carriers are functionalized carriers with different concentrations of additives which are then tested for the viability and long-term stability of certain microbes in these functionalized clay. In one experiment, a microbial inoculant of Bradyrhizobium japonicium (ATCC 35170) was loaded onto two clay-based hybrid carriers 2, 4. The same microbial inoculant was also loaded onto a peat carrier 6 to provide a commercially available baseline to compare against.

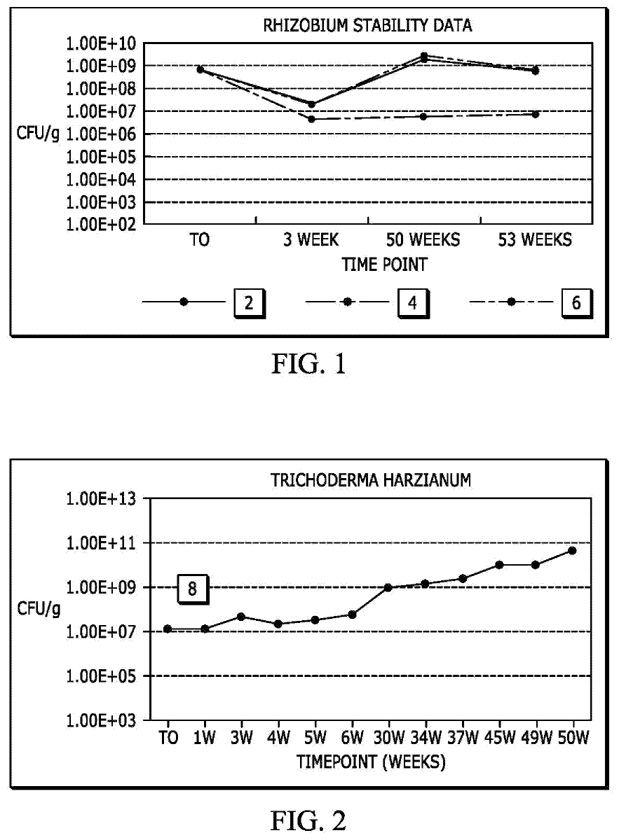

FIG. 1 shows the results of the experiment and demonstrates how the clay-based hybrid carrier provides a healthy environment for the inoculated microbes for an extended period of time, at least up to a year (and likely much longer). The data shows viability of Bradyrhizobium japonicium (ATCC 35170) in the clay-based hybrid carriers 2, 4 is almost two logs more compared to commercially available peat 6, especially as the length of time increases. Viability of microbial cells measured as Colony Forming Unit (CFU) per gram of carrier material tested (CFU/g).

In another experiment, a microbial inoculant of Trichoderma harzianum (ATCC 42835) was tested with a clay-based hybrid carrier 8 especially designed for fungal microbes. clay-based hybrid carrier for an extended period of time, the results of which are shown in FIG. 2. After more than a year the fungus actually grew to nearly 4 logs compared to initial concentrations, and remained stable.

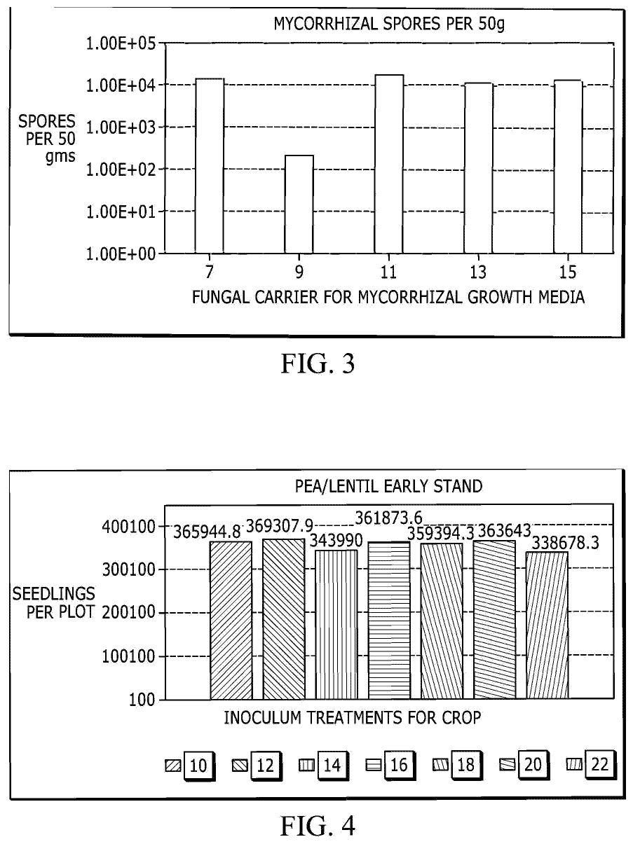

In another experiment, five clay-based hybrid carriers 7, 9, 11, 13, 15 as described herein were used as carriers for the mycorrhizal fungi Glomus intraradices. Infectivity assay such as mean infection percentage can be early colonization indications and are a significant indicator of later spore production. The clay-based hybrid carriers 7, 9, 11, 13, 15 gave a primary infection within 21 days, which was significantly higher when compared to growth of the fungus in control media. While good standard colonization at 21 days starts at around 20% colonization, the clay-based hybrid carriers 7, 9, 11, 13, 15 described herein were at around 45-84% colonization. After three months of growth, the spores were extracted and counted from each fungal carrier 7, 9, 11, 13, 15, the results of which are shown in FIG. 3. Increased spore count was recorded for each fungal carrier 7, 9, 11, 13, 15.

A powdered form of this improved carrier can be used for seed coating and treatment applications. In addition, while this embodiment describes the use of the functionalized carrier platform as a carrier for microbes, it also has a variety of other uses in the agricultural industry, such as a carrier for enzymes, peptides, lysozymes or essential oils for use with crop plants.

Limited, experimental field trials were conducted to determine the efficacy of the clay-based hybrid carriers described above. Several types of carriers were used as a carrier for the bacteria Rhizobium leguminosarum biovar viceae to promote growth in pea/lentil at several locations in eastern Nebraska, Minnesota, Wisconsin, Illinois, and central Michigan. The same types of carriers were used for the bacteria Bradyrhizobium japonicum to promote growth of soybean at the same locations. The plot size was 10 feet×40 feet or 20 feet×80 feet, and each treatment was replicated 3 to 6 times depending on the plot size at each site.

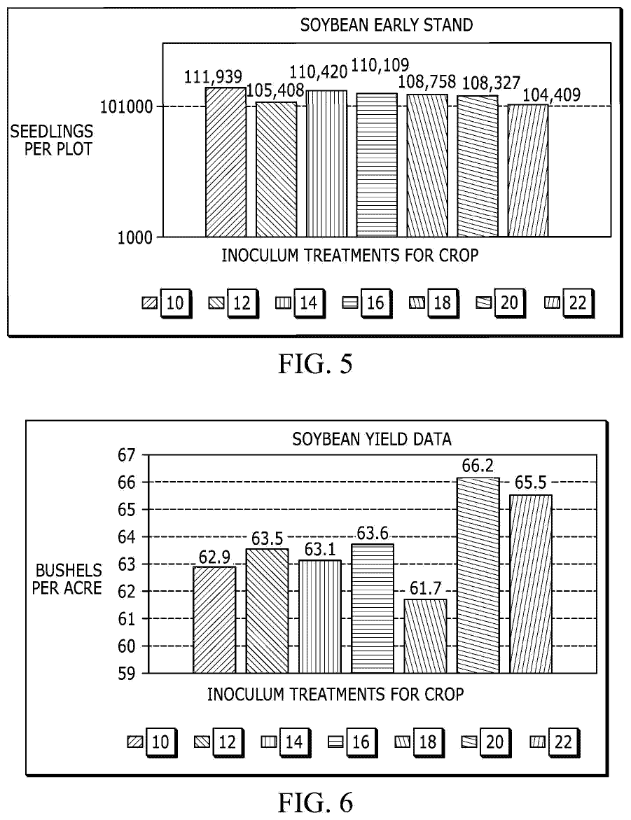

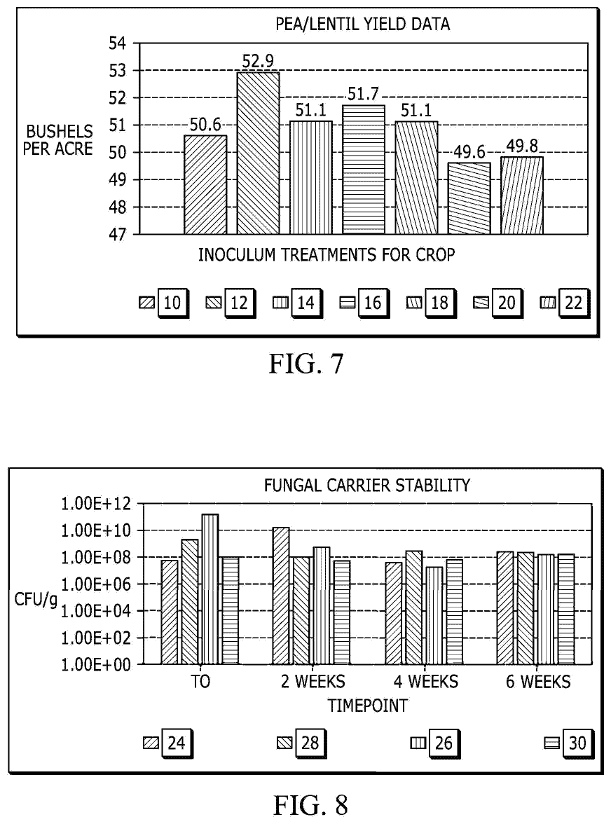

FIGS. 4-7 show some results from these field trials. As can be seen in these figures, seven different types of hybrid carriers were tested: four clay-based hybrid carriers 10, 12, 14, 16 having various formulations all falling within the scope of the examples described above, peat 18, a liquid inoculant 20, and a control 22. FIG. 4 shows the results for stand at 14 days after planting (DAP) for the pea/lentil trials while FIG. 5 shows the results for stand at 14 DAP for the soybean trials. As shown each of the four clay-based hybrid carriers tested demonstrated a numeric increase in stand early in the season when compared to the control. Such a measurement normally indicates at least the potential for increased yields in comparison to the control, as shown in the potential yield charts in FIGS. 6 and 7. In addition, in several instances certain of the clay-based hybrid carriers outperformed peat, which is the most commonly used carrier at this time.

The named inventors conducted another experiment to determine the shelf life of the clay-based hybrid carrier, the results of which are shown in FIG. 7. Commercially used biocontrol fungi Verticillium lecan and Trichodema harzianum were tested, with two clay-based carriers tested for each fungi (24, 26 for Verticillium lecan and 28, 30 for Trichodema harzianum). Both fungi remained relatively stable in the carriers 24, 26, 28, 30 over the course of 6 weeks, with some even increasing in population at times.

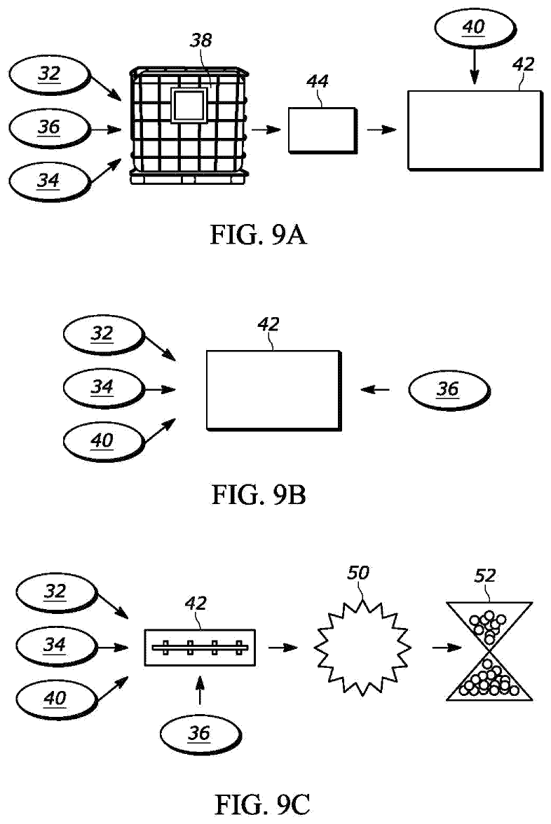

The improved clay-based hybrid carrier may be made using several processes as illustrated in various flow-charts shown in FIGS. 9A-C. For example, in an embodiment, as illustrated in FIG. 9A, a solution 38 containing the carbon source 32, pH modifier 34, and water 36 is applied to the clay/mineral 40 by loading the clay/mineral 40 in granule or pellet form into a mixer 42 and using a sprayer 44 to spray the solution 38 onto the clay/mineral 40 while mixing. Once the solution 38 is properly mixed with the clay/mineral 40, the granules or pellets of the clay/mineral 40 remain with the solution 38 absorbed. The granules or pellets may then be packaged and distributed.

In another embodiment, as illustrated in FIG. 9B, the clay/mineral 40 may be loaded in a powdered or granular form into a mixer, pin mixer, or agglomerator 42 along with the carbon source 32 and pH modifier 34 (if in dry form). As the mixing or compounding begins, 10%-35% of water 36 may be added until the entire mixture agglomerates and forms granules. Alternatively, if the carbon source 32 and pH modifier 34 are in wet form, they may be combined with the water 36 into a single solution before mixing and then the solution may be applied to the clay/mineral 40 during mixing until the mixture agglomerates and forms granules. The granules may then be dried, for example to moisture levels of 4%-10%.

In yet another embodiment, as illustrated in FIG. 9C, the clay/mineral 40 may be provided in a powder form and combined with the carbon source 32 and pH modifier 34 into a blender or mixer 44. Water 36 may optionally be added until the entire mixture agglomerates and forms granules. The mixture may then be freeze dried, for example at −30 to −50 degrees Celsius, as shown at step 50 in FIG. 9C. The mixture may then be ground as shown at step 52 in FIG. 9C until the particle sizes range from 1 micron to 5000 microns. The mixture may then be dry blended until it is homogeneous and then packaged. Such a product may be especially beneficial in agricultural applications.

The foregoing examples have been provided merely for the purpose of explanation and are in no way to be construed as limiting of the present disclosure disclosed herein. While the disclosure has been described with reference to various embodiments, it is understood that the words which have been used herein are words of description and illustration, rather than words of limitation.

Further, although the disclosure has been described herein with reference to particular means, materials and embodiments, the disclosure is not intended to be limited to the particulars disclosed herein; rather, the disclosure extends to all functionally equivalent structures, methods and uses, such as are within the scope of the appended claims. Those skilled in the art, having the benefit of the teachings of this specification, may affect numerous modifications thereto and changes may be made without departing from the scope and spirit of the disclosure in its aspects.

Whereas particular aspects of this disclosure have been described above for purposes of illustration, it will be evident to those skilled in the art that numerous variations of the details of the present disclosure may be made without departing from the disclosure.

While particular preferred embodiments have been shown and described, it is to be understood that the foregoing description is exemplary and explanatory only and is not restrictive of the instant disclosure. Those skilled in the art will appreciate that changes and additions may be made without departing from the instant teachings. For example, the teachings of the instant disclosure may be directed to various combinations and sub-combinations of the disclosed features and/or combinations and sub-combinations of several further features described herein. It is therefore contemplated that any and all modifications, variations, or equivalents of the above-described teachings fall within the scope of the basic underlying principles disclosed above and claimed herein.

1. An improved carrier for sustaining microbes, comprising:
a composition comprising a base material comprising one or more of a clay or mineral, the composition further comprising a carbon source and a pH modifier.

2. The improved carrier of claim 1, wherein the base material comprises one or more of montmorillonites, calcium montmorillonites, attapulgites, palygorskites, sepiolites, kaolins, micas, zeolites, and diatomaceous earth.

3. The improved carrier of claim 1, wherein the carbon source comprises one or more of humic acid, leonardite, biochar, seaweed, corn meal, millet, oats, grain starches, carboxymethyl celluloses, polymers, or other organic cellulose carbohydrates.

4. The improved carrier of claim 1, wherein the pH modifier comprises one or more of salts of calcium, potassium, and sodium.

5. The improved carrier of claim 1, wherein the base material comprises about 85%-99% by weight of the total composition, the carbon source comprising no more than 10% by the weight of the total composition, and the pH modifier comprising no more than 5% of the weight of the total composition.

6. The improved carrier of claim 5, wherein the composition further comprises water, the water comprising no more than 15% by weight of the total composition.

7. The improved carrier of claim 1, wherein the base material comprises a plurality of particles, wherein each of the plurality of particles has a particle size ranging between 30 and 500 microns.

8. The improved carrier of claim 1, wherein the composition comprises a powder.

9. The improved carrier of claim 1, wherein the composition comprises a plurality of pellets.

10. The improved carrier of claim 1, wherein the base material comprises a calcium montmorillonite, the carbon source comprises a humic acid, and the pH modifier comprises a calcium carbonate powder, wherein the base material comprises about 88.5%-92% by weight of the total composition, the carbon source comprises about 2.5%-5% by weight of the total composition, and the pH modifier comprises about 0.5%-1.5% by weight of the total composition, wherein the composition further comprises water, the water comprising about 5% by weight of the total composition.

11. A method of sustaining microbes, comprising:
providing an improved carrier comprising a base material comprising one or more of a clay or mineral, the composition further comprising a carbon source and a pH modifier; and
loading a microbial inoculant onto the carrier, wherein the microbial inoculant comprises a plurality of microbes.

12. The method of claim 11, wherein the microbial inoculant comprises a fully grown broth.

13. The method of claim 11, wherein the microbial inoculant comprises microbial propagules.
